(19) United States
(12) Patent Application Publication
(10) Pub. No.: US 2026/0166264 A1
(43) Pub. Date: Jun. 18, 2026

(54) MEDICAL PACKAGING

(71) Applicant: ConvaTec Limited, Flintshire (GB)

(72) Inventors: Aliaksandr Piashevich, Flintshire (GB); Alice Young, Chester (GB)

(21) Appl. No.: 19/465,959

(22) Filed: Jan. 30, 2026

Related U.S. Application Data
(63) Continuation of application No. PCT/GB2024/051932, filed on Jul. 22, 2024.
(60) Provisional application No. 63/516,559, filed on Jul. 31, 2023.

(30) Foreign Application Priority Data
Sep. 19, 2023 (GB) 2314365.4

(51) Int. Cl. A61M 25/00

(52) U.S. Cl. CPC A61M 25/002; A61M 2202/0014; A61M 2202/0496; A61M 2209/06

(57) ABSTRACT

A catheter packaging defined by at least two walls and having an openable top, wherein the openable top is sealed by a top seal, wherein the two walls extend beyond the top seal forming first and second flaps, each flap having two lateral edges and a top edge, wherein the first flap is provided with a notch on or adjacent to a lateral edge.

### TECHNICAL FIELD OF THE DISCLOSURE

The present disclosure relates to medical packaging. In particular the disclosure concerns packaging for urinary catheter assemblies, and most particularly, but not exclusively packaging for intermittent male urinary catheter assemblies, especially “closed” catheter assemblies.

### BACKGROUND TO THE DISCLOSURE

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows thm to enjoy a relatively normal lifestyle.

It is desirable that catheter assemblies are discreet such that a layperson may not identify them as a packaged catheter assembly at all. This can make a user feel more comfortable carrying and using them. However, along with being compact, the packaged catheter assembly must remain usable such as by providing visual feedback of the drain of fluid from the body. This can make providing a discreet packaged catheter assembly that is also easy to use difficult and so a user may decide to place the packaged catheter assembly in a dedicated bag to hide it. However, this can cause hygiene issues if the bag is not cleaned/replaced regularly and also increase the weight and bulk of the packaged catheter assembly when carried around.

Users can also have problems in opening packaging and accessing its contents. This can happen because the packaging must be robust enough to ensure its contents is not inadvertently released and so opening of the packaging may require careful manipulation as well as a large force. This can mean the user either cannot use the catheter as they cannot access it or that the package is not opened in a controlled manner. This can cause embarrassment for the user but also inconvenience as the catheter may be rendered useless if it is allowed to fall on the floor or otherwise comes into contact with dirt.

It is an object of embodiments of the present disclosure to at least partially overcome or alleviate the above problems and/or to provide an improved packaging for a catheter assembly.

### SUMMARY OF THE DISCLOSURE

In broad terms, the present disclosure concerns a catheter packaging. In addition, the present disclosure also concerns a packaged catheter assembly comprising the catheter packaging and a catheter assembly, wherein the packaging is configured to contain the catheter assembly within it.

According to one broad aspect of the present disclosure there is provided a catheter packaging defined by at least two walls. Preferably the two walls are bonded along a pair of lateral edges by lateral edge bonds. Preferably the packaging has an openable top. Preferably the openable top is sealed by a top seal. The top seal may comprise top bonds. Preferably the top seal meets the lateral edge bonds at a pair of corners. Preferably one or both corners comprise a tear stop.

As such in a preferred broad aspect of the present disclosure there is provided a catheter packaging defined by at least two walls bonded along a pair of lateral edges by lateral edge bonds, the packaging having an openable top, wherein the openable top is sealed by a top seal comprising a top bond which meets the lateral edge bonds at a pair of corners, wherein the one or both corners comprise a tear stop.

Advantageously, by providing a tear stop at the corner manufacture of the packaging is simplified as the need for an additional weld for a tear stop elsewhere (for example of the lateral edges) is removed.

Preferably the corners have an internal angle of no more than 110 degrees.

As such, according to a first aspect of the present disclosure there is provided a catheter packaging defined by at least two walls bonded along a pair of lateral edges by lateral edge bonds, the packaging having an openable top, wherein the openable top is sealed by a top seal comprising a top bond which meets the lateral edge bonds at an internal angle of no more than 110 degrees.

Advantageously, by providing the corners between the top seal and lateral edge bonds with an internal angle of no more than 110 degrees, the corner forms a “tear stop” which provides haptic feedback to the user and restricts the separation of the top bond continuing to the lateral edge bonds without the application of a greater force. That is, the two walls may separate along the top bond but when that separation reaches the corners the resistance increases, requiring the user to actively increase the force applied should they wish to open the bag further. This reduces the risk that the contents may accidentally be spilled. Utilising the corner to form the tear stop also simplifies manufacture as compared to alternatives where a separate tear stop is provided.

The internal angle may be no more than 109 degrees. The internal angle may be no more than 108 degrees. The internal angle may be no more than 107 degrees. The internal angle may be no more than 106 degrees. The internal angle may be no more than 105 degrees. The internal angle may be no more than 104 degrees. The internal angle may be no more than 103 degrees. The internal angle may be no more than 102 degrees. The internal angle may be no more than 101 degrees. The internal angle may be no more than 100 degrees. The internal angle may be no more than 99 degrees. The internal angle may be no more than 98 degrees. The internal angle may be no more than 97 degrees. The internal angle may be no more than 96 degrees. The internal angle may be no more than 95 degrees. The internal angle may be no more than 94 degrees. The internal angle may be no more than 93 degrees. The internal angle may be no more than 92 degrees. The internal angle may be no more than 91 degrees. The internal angle may be no more than 90 degrees.

The internal angle may be between 115 and 95 degrees, preferably between 112.5 and 97.5 degrees, more preferably between 110 and 100 degrees, for example between 107.5 and 102.5 degrees, such as between 106 and 104 degrees, e.g. 105 degrees.

Advantageously angles within these ranges provide a suitable degree of increased resistance to separation.

The force to overcome the tear stop may be at least 1.1 times the force required to overcome the top bond. The force to overcome the tear stop may be at least 1.2 times the force required to overcome the top bond. The force to overcome the tear stop may be at least 1.3 times the force required to overcome the top bond. The force to overcome the tear stop may be at least 1.4 times the force required to overcome the top bond. The force to overcome the tear stop may be at least 1.5 times the force required to overcome the top bond. The force to overcome the tear stop may be at least 1.6 times the force required to overcome the top bond. The force to overcome the tear stop may be at least 1.7 times the force required to overcome the top bond. The force to overcome the tear stop may be at least 1.8 times the force required to overcome the top bond. The force to overcome the tear stop may be at least 1.9 times the force required to overcome the top bond. The force to overcome the tear stop may be at least 2.0 times the force required to overcome the top bond. The force to overcome the tear stop may be at least 2.1 times the force required to overcome the top bond. The force to overcome the tear stop may be at least 2.2 times the force required to overcome the top bond. The force to overcome the tear stop may be at least 2.3 times the force required to overcome the top bond. The force to overcome the tear stop may be at least 2.4 times the force required to overcome the top bond. The force to overcome the tear stop may be at least 2.5 times the force required to overcome the top bond. The force to overcome the tear stop may be at least 2.6 times the force required to overcome the top bond. The force to overcome the tear stop may be at least 2.7 times the force required to overcome the top bond. The force to overcome the tear stop may be at least 2.8 times the force required to overcome the top bond. The force to overcome the tear stop may be at least 2.9 times the force required to overcome the top bond. The force to overcome the tear stop may be at least 3.0 times the force required to overcome the top bond.

Advantageously, the force required to overcome the tear stop as outlined above provide a suitable balance between providing feedback to the user and preventing accidental further opening, but do not prevent further opening if required.

The force required to overcome the tear stop may be at least 0.5 N greater than the force required to overcome the top bond. The force required to overcome the tear stop may be at least 1 N greater than the force required to overcome the top bond. The force required to overcome the tear stop may be at least 2 N greater than the force required to overcome the top bond. The force required to overcome the tear stop may be at least 3 N greater than the force required to overcome the top bond. The force required to overcome the tear stop may be at least 4 N greater than the force required to overcome the top bond. The force required to overcome the tear stop may be at least 5 N greater than the force required to overcome the top bond. The force required to overcome the tear stop may be at least 6 N greater than the force required to overcome the top bond. The force required to overcome the tear stop may be at least 7.5 N greater than the force required to overcome the top bond. The force required to overcome the tear stop may be at least 10 N greater than the force required to overcome the top bond. The force required to overcome the tear stop may be at least 12.5 N greater than the force required to overcome the top bond. The force required to overcome the tear stop may be at least 15 N greater than the force required to overcome the top bond. The force required to overcome the tear stop may be at least 17.5 N greater than the force required to overcome the top bond. The force required to overcome the tear stop may be at least 20 N greater than the force required to overcome the top bond. The force required to overcome the tear stop may be at least 22.5 N greater than the force required to overcome the top bond. The force required to overcome the tear stop may be at least 25 N greater than the force required to overcome the top bond. The force required to overcome the tear stop may be at least 27.5 N greater than the force required to overcome the top bond. The force required to overcome the tear stop may be at least 30 N greater than the force required to overcome the top bond. The force required to overcome the tear stop may be at least 32.5 N greater than the force required to overcome the top bond. The force required to overcome the tear stop may be at least 35 N greater than the force required to overcome the top bond. The force required to overcome the tear stop may be at least 37.5 N greater than the force required to overcome the top bond. The force required to overcome the tear stop may be at least 40 N greater than the force required to overcome the top bond.

The force required to overcome the top bond may be the force required in the steady state, i.e, where separation has already been initiated and is moving along the top bond. Advantageously, the force required to overcome the tear stop as outlined above provide a suitable balance between providing feedback to the user and preventing accidental further opening, but do not prevent further opening if required.

The two walls may comprise a flexible plastics material. For example, polypropylene (PP), polyethylene terephthalate (PET), low density polyethylene (LDPE), metalized polyester (MET PET) or orientated polypropylene (OPP). The two walls may be formed from a single sheet of material.

The lateral edge bond may comprise any one or more of: a weld; mechanical seal; heat seal; pressure seal; adhesive; solvent bond; ultraviolet bond; ultrasonic weld; laser weld; impulse weld; or friction weld. The lateral edge bond may have a width of between 3 and 12 mm, preferably between 4 and 7 mm, more preferably between 4.5 and 5.5 mm, for example 5 mm. The lateral edge bonds may have a constant width. The top seal may comprise any one or more of: a weld; mechanical seal; heat seal; pressure seal; adhesive; solvent bond; ultraviolet bond; ultrasonic weld; laser weld; impulse weld; or friction weld. The top seal may have a width of between 3 and 12 mm, preferably between 4 and 7 mm, more preferably between 4.5 and 5.5 mm, for example 5 mm. The top seal may have a constant width. The top seal and lateral edge bonds may have the same width. The, or part of the lateral edge bonds may be re-sealable, for example through a resealable adhesive, an additional adhesive that is activated prior or during resealing, or a hook and loop/hook and hook fastener. The, or part of the top seal may be re-sealable, for example through a resealable adhesive, an additional adhesive that is activated prior or during resealing, or a hook and loop/hook and hook fastener. The lateral edge bonds and/or top seal may provide a sterile seal. Thus, the lateral edge bonds and/or top seal can be easily constructed to suit the specific needs of the packaging and ensure the contents remains sterile within the packaging.

The lateral edge bonds may form one or more edges of the packaging. A folded edge may comprise the fold between the two walls. All edges other than the folded edge may be formed by bonding. The two walls may comprise a front wall and a rear wall. The folded edge may comprise a base of the packaging. The lateral edge bonds may define a left lateral edge and a right lateral edge. The top seal may define a top edge. The left and right lateral edges may be defined as the left and right edges when viewing the packaging in the closed (folded) configuration with the front wall in front of the rear wall, the base at the bottom and the top edge at the top.

The top bond may be curved. The top bond may have a single continuous curve. The top bond may comprise a pair of bond lines. The pair of bond lines may meet at an apex.

The two walls may be unbonded beyond the top seal. The two walls may be unbonded above the top bond. The two walls may extend beyond the top seal. A portion of each wall above the top seal may form a flap. A portion of each wall beyond the top bond may form a flap. There may be two flaps. A distal end of the flap may define an top edge of the packaging. One or both flaps may be provided with a finger hole. The finger hole may extend through the respective wall. Both finger holes may be arranged above the point where the pair of bond lines meet. Both finger holes may be sized to allow a user's finger to be at least partially inserted through. Both finger holes may be a circle, an oval, an ellipse, a polygon, a square, a rounded square, a stadium, a triangle, a rounded triangle.

The finger holes may have a width defined as the distance between a point closest to the left lateral edge and a point closest to the right lateral edge of at least 20, 30, 40, 50 mm. The finger holes may have a width of no more than 60, 50, 40, 30 mm. Preferably the width of the finger hole is between 15 and 30 mm, most preferably between 15 and 25, for example 20 mm. The width of the finger holes may be between 10% and 30% the width of the packaging, preferably between 15% and 25% the width of the packaging, for example 20%. Having the finger holes sized as such better directs the force applied onto the top seal.

The packaging may have a width, defined as the distance between the left lateral edge and right lateral edge of the packaging, of at least 70, 80, 90 or 100 mm. The packaging may have a width of no more than 200, 180, 160, 140 or 120 mm. Preferably, the width is between 60-120 mm, more preferably between 70 and 110 mm and most preferably about 80 mm. The packaging may have a height defined as the distance between the upper edge and base of at least 160, 170, 180, 190 or 200 mm. The packaging may have a height of no more than 300, 290, 280, 270 or 260 mm. Preferably, the height is between 200-275 mm, more preferably between 240-260 mm and most preferably it is about 255 mm. Thus, the packaging is a compact shape when folded so that it is convenient and discreet for the user to carry, for example it is easier to fit in a bag or pocket.

The ratio between the internal angle and the width of the packaging may be between 0.45 and 2°/mm, preferably between 0.6 and 1.7°/mm, more preferably between 0.75 and 1.5°/mm, for example between 1 and 1.4°/mm, such as between 1.2 and 1.4°/mm, e.g. 1.31°/mm.

The ratio between the internal angle and the width of the packaging may be at least 0.45°/mm, preferably at least 0.75°/mm, more preferably at least 0.90°/mm, most preferably at least 1.05°/mm, for example at least 1.25°/mm.

The lateral edge bonds may comprise a frangible portion. The frangible portion of the lateral edge bonds may be proximate to the top seal. The lateral edge bond may comprise a further tear stop. The further tear stop may delimit the frangible portion of the lateral edge bonds from a non-frangible portion of the lateral edge bonds. The further tear stop may comprise a widened portion of the lateral edge bond. The further tear stop may be at least twice as wide as the lateral edge bond. The further tear stop may have a width of at least 5 mm, 7 mm, 10 mm or 12 mm. The further tear stop may have a width of between 5 and 15 mm, preferably between 7 and 15 mm, more preferably between 10 and 15 mm, for example between 11 and 13 mm, such as 12 mm. The further tear stop may be bonded with a stronger bonding technique than the frangible portion of the lateral edge bond. The further tear stop may require a separation force at least twice the separation force for the frangible portion of the lateral edge bond. The further tear stop may require a separation force at least twice the separation force for the corner tear stop. The top seal may be frangible.

A longitudinal axis may be defined as extending along the centre of the packaging between the top edge and base. A transverse axis may be defined as perpendicular to the longitudinal axis, extending between the right lateral edge and left lateral edge. The packaging may comprise a centreline extending between the top seal and base midway between the left lateral edge and right lateral edge.

The pair of bond lines may meet at a point. By meeting at a point, that is an apex, the bond lines have a tapering point at their uppermost edge, this means the first point to be separated has a very small cross section thereby reducing the force needed to initiate the separation. The pair of bond lines may meet midway between the lateral edges. The pair of bond lines may meet at a centreline. The pair of bond lines may be symmetrical.

The finger holes and the top seal may have a separation of no more than 30 mm, 20 mm, 15 mm, 12 mm, 10 mm, 8 mm. The finger holes and the top seal may have a separation of at least 6 mm, 8 mm, 10 mm, 12 mm. The finger holes and the top seal may have a separation of between 4 and 20 mm, preferably between 6 and 15 mm, more preferably between 7 and 12 mm, for example 10 mm.

Where the packaging comprises two flaps, a first flap may be provided with a notch on or adjacent to a lateral edge.

Accordingly, in a second aspect of the disclosure there is provided a catheter packaging defined by at least two walls and having an openable top, wherein the openable top is sealed by a top seal, wherein the two walls extend beyond the top seal forming first and second flaps, each flap having two lateral edges and a top edge, wherein the first flap is provided with a notch on or adjacent to a lateral edge.

Advantageously, by providing the notch on or adjacent to the lateral edge the overall length of the packaging can be reduced, thereby saving material during manufacture.

The catheter packaging according to the second aspect may optionally include any features, including optional features, of the first aspect and conversely, the catheter packaging of the first aspect may include any optional features of the second aspect.

Each wall may have an interior surface facing the other wall. Each wall may have an opposing exterior surface. The notch may expose an interior surface of the second flap. The interior surface of the second flap may be a different colour to the exterior surface of the first flap. This provides contrast between the two flaps helping to identify the position of the notch to the user.

One, or more, or each flap may be provided with a finger hole. The finger hole may extend through the flap.

Accordingly, in a preferred embodiment there is provided a catheter packaging defined by at least two walls and having an openable top, wherein the openable top is sealed by a top seal, wherein the two walls extend beyond the top seal forming first and second flaps, each flap having two lateral edges and a top edge, wherein the first flap is provided with a notch on or adjacent to a lateral edge, wherein each flap is provided with a finger hole extending through their respective wall.

Advantageously, the provision of finger holes allows users with limited dexterity to open the packaging more easily. The notch allows the user to separate the two flaps and then insert a finger into each finger hole to open the packaging.

In a further preferred embodiment there is provided a catheter packaging defined by at least two walls and having an openable top, wherein the openable top is sealed by a top seal, wherein the two walls extend beyond the top seal forming first and second flaps, each flap having two lateral edges and a top edge, wherein the first flap is provided with a notch on or adjacent to a lateral edge, wherein the top seal comprises a pair of bond lines, the pair of bond lines meeting at an apex, and wherein each flap is provided with a finger hole extending through their respective wall, wherein the finger holes are arranges above the apex where the pair of bond lines meet.

Advantageously, the provision of a top seal provided with an apex, where the finger holes are provided above the apex allows the packaging to be more easily opened once the two flaps have been separated using the notches.

The one, or more, or each finger hole may be a finger hole according to the first aspect. In particular the finger hole(s) may be arranged on the centreline of the packaging. The, or each flap may comprise a pull region. The finger hole may have a top edge proximate to the top edge of the flap. The lateral extent (along the longitudinal axis of the packaging) of the pull region may be defined as the region between the top edge of the finger hole and the top edge of the flap. The lateral extent of the pull region may be at least 8, 10, 12 or 14 mm. The transverse extent (perpendicular to the longitudinal axis of the packaging) may be defined by the transverse extent of the finger hole.

The one, or more, or each flap may have rounded corners between the top edge and lateral edges. The rounded corners may have a radius of curvature of at least 5 mm, preferably at least 7 mm, more preferably at least 9 mm, for example 10 mm.

The notch may be provided outside of the pull region.

In a preferred embodiment there is provided a catheter packaging defined by at least two walls and having an openable top, wherein the openable top is sealed by a top seal, wherein the two walls extend beyond the top seal forming first and second flaps, each flap having two lateral edges and a top edge, wherein the first flap is provided with a notch on or adjacent to a lateral edge, wherein each flap is provided with a finger hole extending through their respective wall, wherein each flap comprises a pull region, the lateral extent of the pull region being defined as a region between a top edge of the finger hole and the top edge of the flap, and the transverse extent being defined by the transverse extent of the finger hole, wherein the notch is arranged outside of the pull region.

Advantageously, providing the notch outside of the pull region reduces the overall size of the flaps as the pull region (where the user applies the force) needs to be sufficiently long in the lateral direct so as to not snap under the force required to open the package and the flap would require further material to extend laterally beyond this distance to allow for the notch, where by moving the notch out of the pull region, the top of the pull region can define the maximum lateral extent of the flap.

The notch may be provided on the top edge of the flap. The notch may be provided on a lateral edge of the flap. The notch may be provided at the corner between the lateral and top edges of the flap. Providing the notch on a corner is advantageous as it is easier to manufacture and provides the notch at the greatest distance from the top seal, increasing the separation between the two flaps it can provide.

A second notch may be provided. The second notch may be provided on the opposite corner to the first notch. The two notches may be provided on the same flap. The two notches may be provided on different flaps.

The notch may have a straight profile. The notch may have a curved profile. The notch may have a radius of curvature of at least 5 mm, preferably at least 10 mm, more preferably at least 15 mm, for example at least 20 mm, i.e. 22 mm.

The notch may have a depth of at least 3 mm, preferably at least 5 mm, more preferably at least 6 mm, for example 7 mm. Such a depth provides a suitable amount of surface area for a user to grip. 55

The packaging may be a sterile packaging. That is when sealed the packaging provides a sterile cavity within.

The packaging may contain a catheter assembly. The catheter assembly may comprise a catheter. The catheter assembly may comprise a fluid collection bag.

As such in a broad aspect of the present disclosure there is provided a packaged catheter assembly comprising a catheter and a fluid collection bag arranged in catheter packaging, the catheter packaging defined by at least two walls bonded along a pair of lateral edges by lateral edge bonds, the packaging having an openable top, wherein the openable top is sealed by a top seal comprising a top bond which meets the lateral edge bonds at a pair of corners, wherein the one or both corners comprise a tear stop.

Accordingly, in a third aspect of the disclosure there is provided a packaged catheter assembly comprising a catheter and a fluid collection bag arranged in catheter packaging, the catheter packaging defined by at least two walls bonded along a pair of lateral edges by lateral edge bonds, the packaging having an openable top, wherein the openable top is sealed by a top seal comprising a top bond which meets the lateral edge bonds at an internal angle of no more than 110 degrees.

The catheter packaging of the third aspect may be a catheter packaging according to the first or second aspects of the disclosure, optionally including any optional features thereof.

Additionally, in a fourth aspect of the disclosure there is provided a packaged catheter assembly comprising a catheter and a fluid collection bag arranged in catheter packaging, the catheter packaging defined by at least two walls and having an openable top, wherein the openable top is sealed by a top seal, wherein the two walls extend beyond the top seal forming first and second flaps, each flap having two lateral edges and a top edge, wherein the first flap is provided with a notch on or adjacent to a lateral edge.

The catheter packaging of the fourth aspect may be a catheter packaging according to the first or second aspects of the disclosure, optionally including any optional features thereof.

The catheter assembly is preferably a closed catheter assembly, that is that the catheter assembly comprises a catheter coupled to a fluid collection bag, such that it is configured to collect and retain fluid released from the body through the catheter. The fluid collected/catheter assembly may then be disposed of by the user when is convenient. Of course, embodiments may also be open catheter assemblies in that fluid is not collected or retained by the catheter assembly.

### DETAILED DESCRIPTION OF THE DISCLOSURE

In order that the disclosure may be more clearly understood one or more embodiments thereof will now be described, by way of example only, with reference to the accompanying drawings, of which:

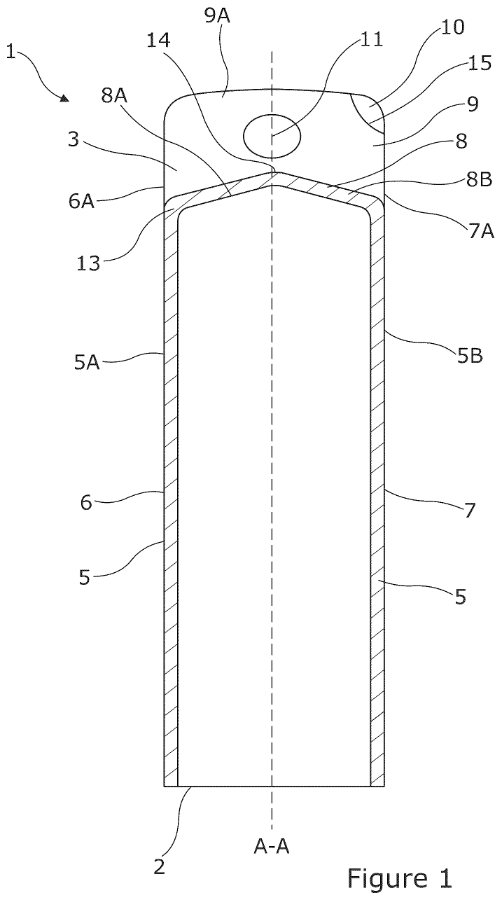

FIG. 1 is a front view of a first embodiment of a catheter packaging;

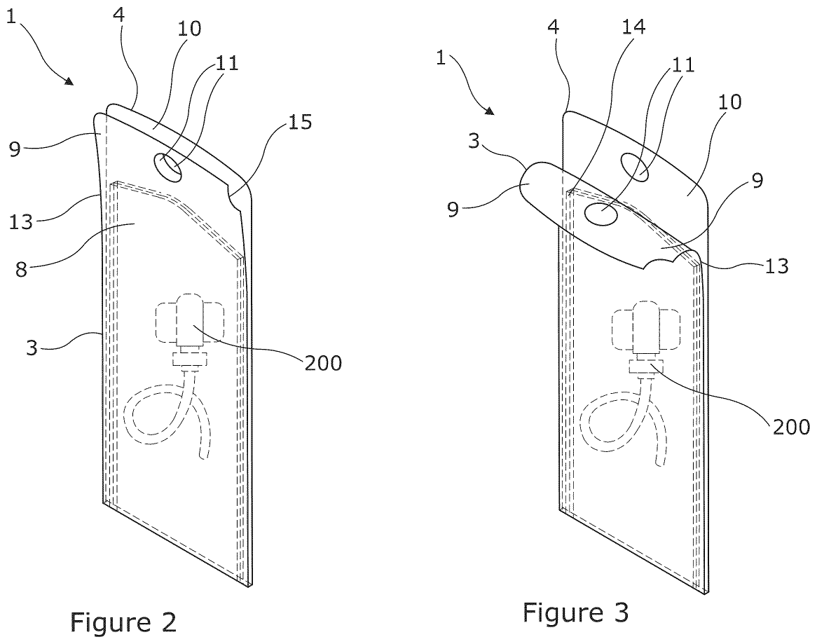

FIG. 2 is a perspective view of the catheter packaging of FIG. 1 in a closed configuration;

FIG. 3 is a perspective view of the catheter packaging of FIG. 1 where the top seal is broken and the top bonds are separated; and

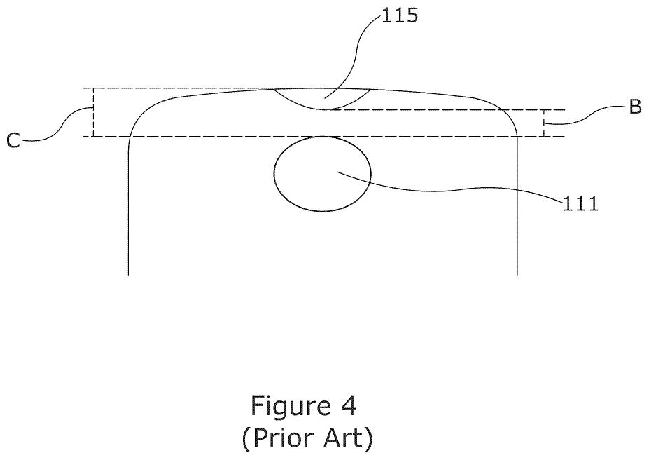

FIG. 4 is a front view of a known catheter packaging.

In the figures, as is conventional, broken lines show hidden features.

Referring to FIG. 1, a first embodiment of a catheter packaging 1 is shown. In this embodiment the packaging 1 is formed from a single piece of material. The packaging 1 is formed by folding this single piece of material along a fold-line 2 spanning the width of the packaging at a point midway along its length to form a front wall 3 and rear wall 4 of the packaging. Lateral edge bonds 5 bond the front 3 and rear walls 4 along their lateral edges and a top seal 8 is provided between the two lateral edge bonds opposite the fold line 2. To aid in the description of the packaging, a longitudinal axis can be considered to extend along a centreline A-A of the packaging from the fold line 2 to the top seal 8.

In this embodiment, the packaging fold-line 2 forms a base edge 2 of the packaging. In this embodiment, the lateral edge bonds 5 form a left lateral edge and a right lateral edge. The left lateral edge 6 and right lateral edge 7 being defined as the right and left sides of the packaging 1 when viewing the packaging 1 with the rear wall 4 behind the front wall 3, the base edge 2 at the bottom of the packaging 1 and the top seal 8 at the top of the packaging 1.

In this embodiment, lateral edge bonds 5 and top seal 8 are formed through heat sealing the front wall 3 to the rear wall 4 with a bond width of 5 mm, but in other embodiments any suitable bonding may be used such as Velcro®, chemical adhesives, chemical bonding, ultrasonic bonding, or mechanical bonding. It is also conceivable that no folds may be provided, for example the front 3 and rear 4 walls may be provided by separate sheets of material, in place of a fold line, the two walls 3,4 may also be joined along the base edge 2 with heat sealing or other suitable bonding.

In this embodiment, the top seal 8 is configured to break to allow the packaging 1 to be opened. Consequently, the packaging 1 has a closed configuration in which it is sealed, and an open configuration in which an openable top is open and a catheter assembly within is accessible, as will be described in greater detail below.

In this embodiment, the packaging 1 is formed from an opaque material and as such provides a discreet package for the packaged catheter assembly which can make the user more comfortable in carrying the packaged catheter assembly in day-to-day life. In addition, the exterior of the packaging 1 may have a textured or printed appearance and feel, such as a matte finish to reduce the impression that it is a medical device.

In this embodiment, a portion of the lateral edges 6,7 of the front and rear walls 3,4 are unbonded beyond the top seal 8. The unbonded portion of the front wall 3 forms a front opening flap 9 and the unbonded portion of the rear wall 4 forms a rear opening flap 10. Both the front opening flap 9 and rear opening flap 10 project from the packaging 1 along an edge corresponding to the top seal 8. Each of the flaps 9, 10 of this particular embodiment are substantially rectangular in shape with lateral edges 6A, 7A parallel to and continuing from a respective lateral edge 6,7 of the packaging and a top edge 9A, 10A. Corners between the lateral edges 6A, 7A and the top edges 9A, 10A are rounded with a radius of curvature of 10 mm.

In this embodiment, at a midpoint of each flap 9,10 there is provided a finger hole 11. Both finger holes 11 have a rounded oval shape, the finger hole 11 may have a width, measured perpendicular to the centreline A-A, of 2 cm, and a height, measured along the centreline A-A, of 1.5 cm. The width of the finger holes 11 thereby less than 20% if the width of the packaging. By providing a finger hole of this width the force applied to the finger holes 11 to break the top seal 8 is more directly focused to the top seal 8, this effect is particularly prevalent where the finger holes 11 have a width of no more than 25% of the width of the packaging. The narrowness of the finger hole thereby reducing the force required and making the packaging 1 easier to open. The separation between the finger holes 11 and the top edge 9A, 10A of their respective opening flaps 9, 10 is 10 mm. The provision of at least 10 mm of material between the finger holes 11 and the top edges 9A, 10A is preferable as it ensures sufficient material to resist tearing when force is applied to the finger holes 11 to open the packaging.

In the present embodiment there is provided on the front opening flap 9 at a corner between the flap lateral edge 7A and the top edge 9A of the front flap 9 a notch 15. The notch 15 is thus a region where the front opening flap 9 has been removed, and the rear opening flap 10 is visible when viewed from the front side, that is to say a peripheral edge of the rear opening flap 10 extends beyond a peripheral edge of the front opening flap 9 in the vicinity of the notch 15. The notch 15 has a curved profile with a radius of curvature of 22 mm. In combination with the rounded corner of the rear opening flap 10 this provided a notch 15 that exposes 7 mm of the rear opening flap 10 at the deepest point, this is sufficient to allow a user to initially separate the two flaps 9, 10 prior to using the finger holes 11. In other embodiments the radius of curvature of either or both the corner and notch 15 may be varied to adjust the size of rear opening flap 10 made accessible.

In further embodiments, an inwardly facing surface of the rear wall may be differently coloured to an outwardly facing surface of the front wall, thereby providing visual contrast to assist the user in locating the notch 15.

In other embodiments, there may be provided two notches, one in each corner of the front opening flap 9, this would allow users with reduced dexterity to preferentially select the side they use.

Advantageously, by providing the notch(es) 15 at a corner(s), the overall length of the packaging can be reduced as compared to known packaging. FIG. 4 shows a packaging where a notch 115 is provided centrally on a top edge directly above a finger hole 111, in order to ensure that pulling on the finger hole does not tear the flap, distance B between the deepest point in the notch and the finger hole 111 is preferably at least 10 mm, however to accommodate this above the finger hole, the flap adjacent to the notch 115 must extend a greater distance C. The depth of the notch 115 is thus C-B and in essence is created by providing additional material adjacent and beyond the notch 115. Moving the notch away from the centre of the top edge thus reduces the overall length of the packaging, thereby reducing the amount of material that is required for manufacture.

In other embodiments the notch may be provided entirely on a lateral edge 6A, 7A or alternatively on a section of the top edge 9A not above the finger hole 11.

The top seal 8 may be provided by two bond lines 8A,B, in this embodiment the left bond line 8A and right bond line 8B are congruent, one being the reflection of the other about the centreline A-A, as such only the left bond alone 8A will be described in detail, and the skilled person will be able to infer the properties of the right bond line 8B accordingly.

The left bond line 8A intersects the left lateral edge bond 5A, and in this embodiment the interface 13 between the two has an abrupt internal angle of 105 degrees, the left bond line 8A extends linearly to the centreline A-A where it meets the right bond line 8B at a point 14.

The provision of an abrupt angle, for example, one with an internal angle of no more than 110°, allows the corners to act as a “tear stop”, a tear stop acts as a point of increased resistance to the separation of the bond between the front 3 and rear 4 walls, this provides feedback to the user and reduces the risk of accidentally opening the packaging too much spilling the contents. However, it also allows the user to apply additional force and continue opening the packaging in a controlled manner should this be desired.

The base 2, lateral edge bonds 5 and top seal 8 together define a sterile cavity within the packaging in which a catheter assembly (not shown) can be stored prior to use.

With reference to FIGS. 2 and 3 the opening of the packaging 1 is described. When the user wishes to use the catheter assembly 200 they use the notch 15 to initially separate the front 9 and rear 10 opening flaps. They then put a finger (or thumb) into each of the finger holes 11 and gripping the opening flaps 9,10 pull the two opening flaps 9,10 apart, as shown in FIG. 2. Starting at the point 14 of the two bond lines 8A,B the front 3 and rear 4 walls begin to separate.

Continued pulling apart of the two tabs 9,10 continues the breaking of the top seal 8 along the bond lines 8A,B, the separation continues until the bond line reaches the corner 13 where the discontinuity at the corner hinders further separation of the join, the user can now access the catheter assembly 200. Once the user has used the catheter assembly it may be returned to the packaging for later disposal. Where the top seal 8 and lateral edge bonds 5 comprise a resealable bond, the packaging may be resealed around the used catheter assembly 200.

In some embodiments, if desired it may be possible to separate the bonds beyond the corner 13. To achieve this a greater force can be applied, this will overcome the increased resistance at the corner 13 and allow the separation to proceed down the lateral edge bonds 5A,B.

The one or more embodiments are described above by way of example only. Many variations are possible without departing from the scope of protection afforded by the appended claims.

1. A catheter packaging defined by at least two walls and having an openable top, wherein the openable top is sealed by a top seal, wherein the two walls extend beyond the top seal forming first and second flaps, each flap having two lateral edges and a top edge, wherein the first flap is provided with a notch on or adjacent to a lateral edge.

2. A catheter packaging according to claim 1 wherein the notch exposes an interior surface of the second flap.

3. A catheter packaging according to claim 1 or 2 wherein each flap is provided with a finger hole extending through their respective wall.

4. A catheter packaging according to claim 3 wherein the separation between the finger hole and the top edge is at least 10 mm.

5. A catheter packaging according to claim 3 or 4 wherein each flap comprises a pull region, the lateral extent of the pull region being defined as the region between a top edge of the finger hole and the top edge of the flap, and the transverse extent being defined by the transverse extend of the finger hole, wherein the notch is arranged outside of the pull region.

6. A catheter packaging according to any preceding claim wherein the notch is provided at a corner between the lateral and top edges of the flap.

7. A catheter packaging according to any preceding claim wherein the length of each flap is less than or equal to 40 mm.

8. A catheter packaging according to any preceding claim wherein the notch has a curved profile.

9. A catheter packaging according to any preceding claim wherein the notch has a radius of curvature of at least 20 mm.

10. A catheter packaging according to any preceding claim wherein the notch has a depth of at least 6 mm.

11. A catheter packaging according to any preceding claim wherein the top seal comprises a pair of bond lines, the pair of bond lines meeting at an apex.

12. A catheter packaging according to claim 11 when dependent, directly or indirectly on claim 3 wherein the finger holes are arranged above the apex where the pair of bond lines meet.

13. A catheter packaging according to any preceding claim wherein the packaging has a width of between 60 and 120 mm.

14. A catheter packaging according to any preceding claim wherein the packaging has a length of between 200 and 275 mm.

15. A catheter packaging according to claim 13 when directly or indirectly dependent upon claim 3 wherein the width of the finger holes is between 15% and 25% the width of the packaging.

16. A catheter packaging according to any preceding claim wherein the packaging is a sterile packaging.

17. A packaged catheter assembly comprising a catheter and a fluid collection bag arranged in catheter packaging, the catheter packaging defined by at least two walls bonded along a pair of lateral edges by lateral edge bonds, the packaging having an openable top, wherein the openable top is sealed by a top seal comprising a top bond which meets the lateral edge bonds at an internal angle of no more than 110 degrees.

18. A packaged catheter assembly according to claim 17 wherein the packaging is a packaging according to any of claims 2 to 16.

19. A packaged catheter assembly according to claim 17 or 18 wherein the catheter is a closed catheter.
